(19) United States
(12) Patent Application Publication
(10) Pub. No.: US 2007/0203237 A1
(43) Pub. Date: Aug. 30, 2007

(54) Method for preventing or treating obesity

(76) Inventors: J. Thomas Brenna, Ithaca, NY (US); Kristin Morris, Evansville, IN (US); Joshua C. Anthony, Evansville, IN (US); Steven C. Rumsey, Curitiba (BR)

Correspondence Address: Richard D. Schmidt;Bristol-Myers Squibb Company, 2400 West Lloyd Expressway, Evansville, IN, 47721-0001, US

(21) Appl. No.: 11/711,196

(22) Filed: Feb. 27, 2007

Related U.S. Application Data
(60) Provisional application No. 60/777,345, filed on Feb. 28, 2006.

(51) Int. Cl. A61K 31/202; A61K 47/00

(52) U.S. Cl. 514/560; 424/439

(57) ABSTRACT

The present invention is directed to a novel method for preventing or treating obesity in a subject. The method comprises administration of a therapeutically effective amount of DHA and ARA, alone or in combination with one another, to the subject.

This application claims the priority benefit of U.S. Provisional Application 60/677,345 filed Feb. 28, 2006 which is incorporated by reference herein in its entirety.

### BACKGROUND OF THE INVENTION

(1) Field of the Invention

The present invention relates generally to a method for preventing or treating obesity.

(2) Description of the Related Art

In the United States, more than 25% of adults and more than 14% of children and adolescents are obese. Obesity is a medical condition that takes various factors into account, such as body mass index (BMI) and waist circumference. For example, if a man has a BMI over 30 and has a waist circumference that is greater than 40 inches, he may be considered obese. Obesity is also determined based on a comparison of the amount of adipose tissue, a specialized connective tissue that functions as the major storage site for fat, versus lean muscle in the body. Obesity causes significant morbidity, decreased life expectancy, and has been shown to contribute to high blood pressure, breathing problems, stroke, heart disease, diabetes, hyperlipidemia, high cholesterol levels, gallbladder disease, gout, some types of cancer, and osteoarthritis.

There is evidence that obesity tracks from infancy to adulthood. Zive, M. M., et al., Infant-feeding Practices and Adiposity in 4-y-old Anglo-and Mexican-Americans, Am. J. Clin. Nutr. 55:1104-1108 (1992). In fact, studies have found that one-third of obese adults were obese children and 50% of obese adolescents were obese in infancy. Mulhins, A. G., The Prognosis in Juvenile Obesity, Arch. Dis. Childhood 33:307-314 (1958); Poskitt, E. M. E., The Fat Child. Clin. Paediatr. Endocrin. 141-158 (1981).

Though adult obesity can be easily measured through BMI and waist circumference, the same does not apply to infants or children. Researchers and clinicians agree that a body composition assessment, which is a measure of the amount of body mass that is present as fat, bone, and lean muscle, provides a much better gauge of infant or child growth and nutritional status than length and weight measurements. Thus, the best way to prevent the onset of obesity in childhood, adolescence or adulthood may be to improve body composition in infancy.

Therefore, it would be beneficial to provide a composition that can improve the body composition of infants and children and thereby prevent the onset of obesity in childhood, adolescence or adulthood. In addition, it would be beneficial to provide an infant formula or nutritional supplement containing such a composition in order to improve the body composition of infants and children.

### SUMMARY OF THE INVENTION

Briefly, the present invention is directed to novel method for preventing or treating obesity in a subject, the method comprising administering to the subject a therapeutically effective amount of DHA or ARA, alone or in combination with one another. The subject may be an infant or a child.

The invention is also directed to a novel method for increasing the lean muscle mass and decreasing the adipose tissue of a subject, the method comprising administering to the subject a therapeutically effective amount of DHA or ARA, alone or in combination with one another. In addition, the invention is directed to a method for upregulating the expression of IL-15 in a subject's skeletal muscle, the method comprising administering to the subject a therapeutically effective amount of DHA or ARA, alone or in combination with one another. The invention is additionally directed to a method for downregulating the expression of IL-15 in a subject's subcutaneous adipose tissue, the method comprising administering to the subject a therapeutically effective amount of DHA or ARA, alone or in combination with one another.

Further, the invention is directed to a method for upregulating the expression of adiponectin in a subject's skeletal muscle, the method comprising administering to the subject a therapeutically effective amount of DHA or ARA, alone or in combination with one another. In addition, the invention is directed to a method for downregulating the expression of the hepatic leptin receptor in a subject, the method comprising administering to the subject a therapeutically effective amount of DHA or ARA, alone or in combination with one another.

Among the several advantages found to be achieved by the present invention, is that it prevents the onset of or treats obesity. The invention increases the amount of lean muscle in the body and decreases the amount of adipose tissue. As such, the invention may also prevent the occurrence of many diseases and disorders associated with obesity.

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

As used herein, the term “upregulate” means a positive regulatory effect on the expression of genes.

The term “downregulate” means a negative regulatory effect on the expression of genes.

As used herein the term “expression” means the conversion of genetic information encoded in a gene into messenger RNA (mRNA), transfer RNA (tRNA) or ribosomal RNA (rRNA) through transcription.

The terms “therapeutically effective amount” refer to an amount that results in an improvement or remediation of the disease, disorder, or symptoms of the disease or condition.

The term “infant” means a postnatal human that is less than about 1 year of age.

The term “child” means a human that is between about 1 year and 12 years of age. In some embodiments, a child is between the ages of about 1 and 6 years. In other embodiments, a child is between the ages of about 7 and 12 years.

As used herein, the term “infant formula” means a composition that satisfies the nutrient requirements of an infant by being a substitute for human milk. In the United States, the contents of an infant formula are dictated by the federal regulations set forth at 21 C.F.R. Sections 100, 106, and 107. These regulations define macronutrient, vitamin, mineral, and other ingredient levels in an effort to stimulate the nutritional and other properties of human breast milk.

In accordance with the present invention, the inventors have discovered a novel method for preventing or treating obesity in a subject which comprises administering a therapeutically effective amount of docosahexaenoic acid (DHA) and arachidonic acid (ARA) to the subject.

In fact, it has been shown in the present invention that the administration of DHA or ARA, alone or in combination with one another, increases the expression of interleukin-15 (IL-15) in skeletal muscle and decreases the expression of IL-15 in subcutaneous adipose tissue, indicating that the administration of DHA or ARA, alone or in combination with one another, contribute to altering the body composition of an infant or child to have more lean muscle and less fatty adipose tissue.

IL-15 is a cytokine which is highly expressed in skeletal muscle tissue, and which has anabolic effects on skeletal muscle protein. It stimulates skeletal muscle fiber protein synthesis and inhibits protein degradation. Quinn, L. S., et al., Interleukin-15: A Novel Anabolic Cytokine for Skeletal Muscle, Endocrinol. 136:(8)3669-3672 (1995). The administration of IL-15 has also been shown to inhibit white adipose tissue deposition, possibly having a direct effect on such tissue. Alvarez, B., et al., Effects of Interleukin-15 (IL-15) on Adipose Tissue Mass in Rodent Obesity Models: Evidence for Direct IL-15 Action on Adipose Tissue, Biochimica et Biophysica Acta 1570:33-37 (2002).

By stimulating muscle growth and inhibiting adipose tissue growth, the method of the present invention may alter body composition and may be useful in treating obesity. Id. In fact, it has been suggested that alterations in IL-15 receptors could be responsible for some types of obesity. Id. Thus, the effects of DHA or ARA, alone or in combination with one another, on the expression of IL-15 are useful in altering the body composition of infants and children and possibly preventing obesity later in life.

The present invention has also been shown to increase the expression of adiponectin receptor-2 in skeletal muscle. Adiponectin is a protein hormone produced and secreted exclusively by adipose tissue that regulates the metabolism of lipids and glucose. It mediates increased activated protein kinase (AMPK) and peroxisome proliferator-activated receptor (PPAR)-60 ligand activities as well as fatty acid oxidation and glucose uptake by full length adiponectin. Increased expression of adiponectin in skeletal muscle increases skeletal muscle fatty acid oxidation.

Levels of the hormone are inversely correlated with body mass index and obesity. Thus, it is has been suggested that an increased expression of adiponectin could prevent or treat obesity. Haluzik, M., et al., Adiponectin and Its Role in the Obesity-Induced Insulin, Physiol. Res. 53:123-129 (2004). Because the present invention has shown that DHA or ARA, alone or in combination with one another, increase the expression of adiponectin receptor-2 in skeletal muscle, thereby increasing the levels of adpionectin, the method of the present invention is useful in altering body composition and preventing or treating obesity.

The present invention has additionally shown that DHA or ARA, alone or in combination with one another, supplementation decreases expression of the hepatic leptin receptor. Leptin is a hormone produced by white adipose tissue that is involved in energy metabolism and body weight regulation. Leptin operates as a circulating factor that sends a satiety signal to the hypothalamus, thereby suppressing appetite. It has also been shown that leptin increases energy expenditure, measured as increased oxygen consumption, higher body temperatures, and loss of adipose tissue. Thus, in individuals that do not have any genetic defects on the obese (ob) gene, which encodes leptin, increased levels of circulating leptin are correlated with less adipose tissue.

Data suggests that the liver is the primary source of soluble circulating leptin receptor (sOb-R), which sequesters free leptin and limits leptin action. The method of the present invention has shown that DHA or ARA, alone or in combination with one another, may downregulate the expression of the leptin receptor in the liver. By downregulating the expression of the leptin receptor, more leptin remains in circulation, thereby contributing to a decrease in adipose tissue.

In the present invention, the administration of DHA or ARA, alone or in combination with one another, to infants and children has been shown to alter body composition toward having greater amounts of lean muscle and a lesser amount of adipose tissue. DHA and ARA are long chain polyunsaturated fatty acids (LCPUFA) which have previously been shown to contribute to the health and growth of infants. Specifically, DHA and ARA have been shown to support the development and maintenance of the brain, eyes and nerves of infants. Birch, E., et al., A Randomized Controlled Trial of Long-Chain Polyunsaturated Fatty Acid Supplementation of Formula in Term Infants after Weaning at 6 Weeks of Age, Am. J. Clin. Nutr. 75:570-580 (2002). Clandinin, M., et al., Formulas with Docosahexaenoic Acid (DHA) and Arachidonic Acid (ARA) Promote Better Growth and Development Scores in Very-Low-Birth-Weight Infants (VLBW), Pediatr. Res. 51:187A-188A (2002). DHA and ARA are typically obtained through breast milk in infants that are breast-fed. In infants that are formula-fed, however, DHA and ARA must be supplemented into the diet.

While it has been shown that DHA and ARA are beneficial to the development of brain, eyes and nerves in infants, DHA and ARA have not previously been shown to have any effect on preventing or treating obesity. The positive effects of DHA and ARA on the prevention and treatment of obesity were surprising and unexpected.

In some embodiments of the present invention, the subject is in need of the prevention or treatment of obesity. The subject may be at risk due to genetic predisposition, diet, lifestyle, diseases, disorders, and the like. In certain embodiments, the subject is an infant or child. In these embodiments, the infant or child may be in need of the prevention or treatment of obesity.

In the present invention, the form of administration of DHA and ARA is not critical, as long as a therapeutically effective amount is administered to the subject. In some embodiments, the DHA and ARA are administered to a subject via tablets, pills, encapsulations, caplets, gelcaps, capsules, oil drops, or sachets. In another embodiment, the DHA and ARA are added to a food or drink product and consumed. The food or drink product may be a children's nutritional product such as a follow-on formula, growing up milk, or a milk powder or the product may be an infant's nutritional product, such as an infant formula.

In certain embodiments, the subject is an infant. In these embodiments, the DHA or ARA, alone or in combination with one another, can be supplemented into an infant formula which can then be fed to the infant.

In an embodiment, the infant formula for use in the present invention is nutritionally complete and contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, amino acids, and the like. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, canola oil, corn oil, soybean oil, palmolein, coconut oil, medium chain triglyceride oil, high oleic sunflower oil, high oleic safflower oil, and the like.

Conveniently, commercially available infant formula can be used. For example, Enfalac, Enfamil®, Enfamil® Premature Formula, Enfamil® with Iron, Lactofree®, Nutramigen®, Pregestimil®, and ProSobee® (available from Mead Johnson & Company, Evansville, Ind., U.S.A.) may be supplemented with suitable levels of DHA or ARA, alone or in combination with one another, and used in practice of the method of the invention. Additionally, Enfamil® LIPIL®, which contains effective levels of DHA and ARA, is commercially available and may be utilized in the present invention.

The method of the invention requires the administration of DHA or ARA, alone or in combination with one another. In this embodiment, the weight ratio of ARA:DHA is typically from about 1:3 to about 9:1. In one embodiment of the present invention, this ratio is from about 1:2 to about 4:1. In yet another embodiment, the ratio is from about 2:3 to about 2:1. In one particular embodiment the ratio is about 2:1. In another particular embodiment of the invention, the ratio is about 1:1.5. In other embodiments, the ratio is about 1:1.3. In still other embodiments, the ratio is about 1:1.9. In a particular embodiment, the ratio is about 1.5:1. In a further embodiment, the ratio is about 1.47:1.

In certain embodiments of the invention, the level of DHA is between about 0.0% and 1.00% of fatty acids, by weight. Thus, in certain embodiments, the ARA alone may treat or reduce obesity.

The level of DHA may be about 0.32% by weight. In some embodiments, the level of DHA may be about 0.33% by weight. In another embodiment, the level of DHA may be about 0.64% by weight. In another embodiment, the level of DHA may be about 0.67% by weight. In yet another embodiment, the level of DHA may be about 0.96% by weight. In a further embodiment, the level of DHA may be about 1.00% by weight.

In embodiments of the invention, the level of ARA is between 0.0% and 0.67% of fatty acids, by weight. Thus, in certain embodiments of the invention, DHA alone may treat or reduce obesity. In another embodiment, the level of ARA may be about 0.67% by weight. In another embodiment, the level of ARA may be about 0.5% by weight. In yet another embodiment, the level of DHA may be between about 0.47% and 0.48% by weight.

The effective amount of DHA in an embodiment of the present invention is typically from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day.

The effective amount of ARA in an embodiment of the present invention is typically from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

The amount of DHA in infant formulas for use in the present invention typically varies from about 2 mg/100 kilocalories (kcal) to about 100 mg/100 kcal. In another embodiment, the amount of DHA varies from about 5 mg/100 kcal to about 75 mg/100 kcal. In yet another embodiment, the amount of DHA varies from about 15 mg/100 kcal to about 60 mg/100 kcal.

The amount of ARA in infant formulas for use in the present invention typically varies from about 4 mg/100 kilocalories (kcal) to about 100 mg/100 kcal. In another embodiment, the amount of ARA varies from about 10 mg/100 kcal to about 67 mg/100 kcal. In yet another embodiment, the amount of ARA varies from about 20 mg/100 kcal to about 50 mg/100 kcal. In a particular embodiment, the amount of ARA varies from about 25 mg/100 kcal to about 40 mg/100 kcal. In one embodiment, the amount of ARA is about 30 mg/100 kcal.

The infant formula supplemented with oils containing DHA and ARA for use in the present invention may be made using standard techniques known in the art. For example, an equivalent amount of an oil which is normally present in infant formula, such as high oleic sunflower oil, may be replaced with DHA and ARA.

The source of the ARA and DHA can be any source known in the art such as marine oil, fish oil, single cell oil, egg yolk lipid, brain lipid, and the like. The DHA and ARA can be in natural form, provided that the remainder of the LCPUFA source does not result in any substantial deleterious effect on the infant. Alternatively, the DHA and ARA can be used in refined form.

The LCPUFA source may or may not contain eicosapentaenoic acid (EPA). In some embodiments, the LCPUFA used in the invention contains little or no EPA. For example, in certain embodiments that the infant formulas used herein contain less than about 20 mg/100 kcal EPA; in some embodiments less than about 10 mg/100 kcal EPA; in other embodiments less than about 5 mg/100 kcal EPA; and in still other embodiments substantially no EPA.

Sources of DHA and ARA may be single cell oils as taught in U.S. Pat. Nos. 5,374,657, 5,550,156, and 5,397,591, the disclosures of which are incorporated herein by reference in their entirety.

In an embodiment of the present invention, DHA or ARA, alone or in combination with one another, are supplemented into the diet of an infant from birth until the infant reaches about one year of age. In a particular embodiment, the infant may be a preterm infant. In another embodiment of the invention, DHA or ARA, alone or in combination with one another, are supplemented into the diet of a subject from birth until the subject reaches about two years of age. In other embodiments, DHA or ARA, alone or in combination with one another, are supplemented into the diet of a subject for the lifetime of the subject. Thus, in particular embodiments, the subject may be a child, adolescent, or adult.

In an embodiment, the subject of the invention is a child between the ages of one and six years old. In another embodiment the subject of the invention is a child between the ages of seven and twelve years old. In particular embodiments, the administration of DHA to children between the ages of one and twelve years of age is effective in treating or preventing obesity. In other embodiments, the administration of DHA and ARA to children between the ages of one and twelve years of age is effective in treating or preventing obesity.

In certain embodiments of the invention, DHA or ARA, alone or in combination with one another, are effective in treating or preventing obesity in an animal subject. The animal subject may be one that is in need of such prevention or treatment. The animal subject is typically a mammal, which may be domestic, farm, zoo, sports, or pet animals, such as dogs, horses, cats, cattle, and the like.

The present invention is also directed to the use of DHA or ARA, alone or in combination with one another, for the preparation of a medicament for treatment or prevention of obesity. In this embodiment, the DHA or ARA, alone or in combination with one another, may be used to prepare a medicament for treatment or prevention of obesity in any human or animal neonate. In some embodiments, the animal is in need of treatment or prevention of obesity.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLE 1

This example describes the results of DHA and ARA supplementation in improving body composition.

Methods

Animals

All animal work took place at the Southwest Foundation for Biomedical Research (SFBR) located in San Antonio, Tex. Animal protocols were approved by the SFBR and Cornell University Institutional Animal Care and Use Committee (IACUC). Animal characteristics are summarized in Table 1.

TABLE 1
Baboon Neonate Characteristics

| | |
|---|---|
| Number of animals | 14 |
| Gender | 10 female, 4 male |
| Conceptional age at delivery (days) | 181.8 ± 6.2 |
| Birth weight (g) | 860.3 ± 150.8 |
| Weight at 12 weeks (g) | 1519.1 ± 280.7 |
| Weight gain (g) | 658.8 ± 190.4 |

Fourteen pregnant baboons delivered spontaneously around 182 days gestation. Neonates were transferred to the nursery within 24 hours of birth and randomized to one of three diet groups. Animals were housed in enclosed incubators until 2 weeks of age and then moved to individual stainless steel cages in a controlled access nursery. Room temperatures were maintained at temperatures between 76° F. to 82° F., with a 12 hour light/dark cycle. They were fed on experimental formulas until 12 weeks of life.

Diets

Animals were assigned to one of the three experimental formulas, with LCPUFA concentrations presented in Table 2.

TABLE 2
Formula LCPUFA composition

| | C | L | L3 |
|---|---|---|---|
| DHA (%, w/w) | 0 | 0.42 ± 0.02 | 1.13 ± 0.04 |
| DHA | 0 | 21.3 ± 1.0 | 62.8 ± 1.9 |
| (mg/100 kcal) | | | |
| ARA (%, w/w) | 0 | 0.77 ± 0.02 | 0.71 ± 0.01 |
| ARA | 0 | 39.4 ± 0.9 | 39.2 ± 0.7 |
| (mg/100 kcal) | | | |

Target concentrations were set as shown in brackets and diets were formulated with excess to account for analytical and manufacturing variability and/or possible losses during storage. Control (C) and L, moderate DHA formula, are the commercially available human infant formulas Enfamil® and Enfamil LIPIL®, respectively. Formula L3 had an equivalent concentration of ARA and was targeted at three-fold the concentration of DHA.

Formulas were provided by Mead Johnson & Company (Evansville, Ind.) in ready-to-feed form. Each diet was sealed in cans assigned two different color-codes to mask investigators. Animals were offered 1 ounce of formula four times daily at 07:00, 10:00, 13:00 and 16:00 with an additional feed during the first 2 nights. On day 3 and beyond, neonates were offered 4 ounces total; when they consumed the entire amount, the amount offered was increased in daily 2 ounce increments. Neonates were hand fed for the first 7-10 days until independent feeding was established.

Growth

Neonatal growth was assessed using body weight measurements, recorded two or three times weekly. Head circumference and crown-rump length data were obtained weekly for each animal. Organ weights were recorded at necropsy at 12 weeks.

Sampling

Animals were anesthetized, and euthanized by exsanguination at 84.57±1.09 days. Blood was collected in EDTA-containing Vacutainer tubes, and red blood cells (RBC) and plasma were separated by centrifugation. Eyes and one brain hemisphere were removed and immediately dissected. Central nervous system (CNS) structures were dissected by an experienced neurologist, weighted, flash frozen in liquid nitrogen, and stored at −80° C. until they were analyzed in their entirety. Retina and one gram samples of left ventricle and right liver lobe were removed and treated similarly.

Tissues were collected from the skeletal muscle, subcutaneous and visceral adipose tissue, and liver, and isolated for DNA microarray expression analysis.

Analyses

Total lipids were extracted from tissue homogenates using the Bligh and Dyer method. Fatty acid methyl esters (FAME) were prepared using sodium hydroxide and 14% boron-trifluoride (BF₃) in methanol, and were analyzed by gas chromatography (HP 5890; BPX-70 column, SGE, Austin, Tex.), using H₂ carrier gas as described previously. Fatty acid (FA) identities were determined by covalent adduct chemical ionization tandem mass spectrometry and then quantified using methyl heptadecanoate as an internal standard and response factors derived from an equal weight FAME mixture. FA concentrations are expressed as percent weight of total fatty acids from 14 to 24 carbons.

Statistics

Data are expressed as mean±SD. Statistical analysis was conducted using analysis of variance (ANOVA) to test the hypothesis of equivalent means for measures taken at 12 weeks, and Tukey's correction was used to control for multiple comparisons. Formula consumption, body weight, head circumference, and crown-rump length changes over time were tested with a random coefficient regression model to compare LCPUFA groups (L, L3) to control (C). Analysis were performed using SAS for Windows 9.1 (SAS Institute, Cary, N.C.) with significance declared at p<0.05.

Results

Growth

There were no significant differences in formula consumption between LCPUFA groups and the C group over time (p=0.64). Similarly, no significant changes over time were found for body weight (BW, p=0.47), head circumference (p=0.68), crown-rump length (CRL, p=0.38), or the ratio BW/CRL (p>0.50) (data not presented). There were no significant differences in the 12 week data for these anthropometry measures. There were no significant differences and no trends in the 12 week organ weights, expressed as a percent of body weight (BW), for brain, liver, thymus, spleen, heart, lungs, the right kidney, or the pancreas.

Liver and Heart Fatty Acids

Increasing formula DHA significantly elevated liver DHA concentrations; the L and L3 groups had 2.2 and 3.6-fold more DHA than the C group, respectively. In contrast to DHA, dietary ARA increased liver levels in the L group; ARA dropped 14.3% from the L to L3 group. The concentrations of the ARA elongation product, adrenic acid (AdrA), were significantly higher in the C group (0.99±0.13%) relative to L and L3. A similar, but non-significant trend was observed for docosapentaenoic acid (DPA) n-6; levels were highest in C animals, followed by the L and L3 groups. DPAn-3 concentrations dropped 2-fold for LCPUFA animals compared to controls. DPAn-6/DHA was significantly elevated for the C and L groups, compared to L3, by 4.6 and 14 fold. Increases in LCPUFA were compensated by decreases in total monounsaturated fatty acids (MUFA) and linoleic acid (LA, 18:2n-6), but not total saturated fatty acids (SFA).

As with the liver, heart DHA increased in the L and L3 groups, 2.8 and 3.9 fold, respectively, while DPAn-3 dropped significantly. The increases in DHA appear to be at the expense of SFA, though the decrease in SFA from C to L to L3 did not reach statistical significance. Linoleic acid decreased from C to L but L and L3 were not different.

RBC and Plasma Fatty Acids

Supplementation significantly elevated RBC DHA for L and L3 groups by 3.8- and 4.6-fold, compared to controls. A similar trend was observed in plasma, DHA increased by 4.6- and 7.5-fold for the LCPUFA supplemented groups, L and L3. While ARA significantly increased from C to L for RBC, ARA levels declined from the L to the L3 group. A consistent but non-significant trend is present for ARA plasma concentrations, with a moderate increase from C (5.36±1.00) to L (10.06±0.99) and an intermediate level in L3 (7.79±0.84). AdrA is a minor component but did respond to diets in both RBC and plasma, where it decreased significantly in the L3 group compared to the C and L groups. DPAn-6 concentrations were significantly higher in RBC of controls. DPAn-3 levels were higher in the C group compared to the L and L3 groups in both RBC and plasma measurements. The DPAn-6/DHA ratio was significantly greater for control and L animals compared to the L3 group, approximately by 4- and 10-fold.

Retina Fatty Acids

Changes in retinal DHA due to dietary LCPUFA did not reach significance, though the L and L3 group means were greater than the C group by amounts similar to previous reports. ARA concentrations were not influenced by formula composition. DPAn-6 concentrations were significantly higher in controls compared to the highest supplemented group, L3. Levels of DPAn-3 increased with dietary LCPUFA, with L3 significantly elevated compared to the C group. The DPAn-6/DHA index for C and L groups were 3.6-fold higher than the high DHA formula group, L3.

CNS Fatty Acids

DHA concentrations significantly increased with higher levels of formula DHA in the cerebral cortex precentral gyrus, the primary motor cortex region. Supplementation improved DHA levels by 24% and 43% compared to controls in the L and L3 groups, respectively, and the difference between L and L3 was statistically significant. LCPUFA supplementation also significantly increased DHA in frontal cortex by 30% and 41% in the L and L3 groups, respectively, compared to controls, however the difference between L and L3 was borderline significant (p=0.10).

Formula DHA increased DHA in the basal ganglia regions globus pallidus and caudate, and in the midbrain regions superior colliculus and inferior colliculus, however there were no detectable differences between L and L3 groups. The non-significant trends in putamen and amygdala were consistent with this pattern. DPAn-6 decreased significantly and consistently from C to L to L3 in all CNS regions.

With the exception of two CNS regions, dietary manipulation had little influence on ARA levels. Levels of ARA in globus pallidus and superior colliculus were highest in the L formula group, but significantly declined 10% with additional formula DHA.

Similar results for n-3 sufficiency indices were obtained in all brain regions. The DPAn-6/DHA ratio was significantly elevated for C compared to the high formula DHA group, L3, in all CNS regions. The L and L3 groups were significantly different in frontal lobe, globus pallidus, caudate, and inferior colliculus. C and L groups were consistently elevated by 2- to 5-fold, respectively, compared with the L3 group.

Body Composition

The results of the study show that during the early postnatal weeks, supplementation at levels of 0.33% DHA/0.67% ARA and 1.00% DHA/0.67% ARA increased the expression of IL-15 in skeletal muscle and decreased the expression of IL-15 in subcutaneous adipose tissue when compared to an unsupplemented control group. The effects of DHA and ARA on IL-15 expression suggest cross-talk between skeletal muscle and adipose tissue metabolism. DHA and ARA supplementation can promote mobilization of adipose tissue lipid stores while also favorably influencing skeletal muscle protein synthesis and accretion. In addition, supplementation with DHA and ARA increased the expression of adiponectin in skeletal muscle and decreased the expression of the hepatic leptin receptor. These results are shown in Table 3.

TABLE 3¹

| | | Experimental Groups | | |
|---|---|---|---|---|
| Tissue | Gene | Control | L1 | L3 |
| Skeletal Muscle | Adiponectin | 9.029 | 9.256 | 9.827 |
| | Receptor 2 | | | |
| Skeletal Muscle | Interleukin-15 | 2.987 | 3.047 | 3.533 |
| Subcutaneous Adipose | Interleukin-15 | 6.44 | 5.669 | 3.556 |
| Tissue | | | | |
| Liver | Fatty Acid | 7.479 | 6.40 | 6.278 |
| | Desaturase-1 | | | |
| Liver | Fatty Acid | 4.453 | 3.606 | 3.619 |
| | Desaturase-2 | | | |
| Liver | Sterol-CoA | 5.622 | 4.324 | 3.727 |
| | Desaturase | | | |
| Liver | Sterol | 4.362 | 4.349 | 3.407 |
| | Regulatory | | | |
| | Element Binding | | | |
| | Protein-2 | | | |
| Liver | Leptin Receptor | 6.295 | 5.792 | 5.746 |

¹Values are Log 2 base values. Thus, a 2-fold change on a log 2 scale represents a four fold change on a linear scale. For example, IL-15 expression in subcutaneous adipose tissue was nearly 8-fold higher on a linear scale in the control group compared with the L3 group (6.444 vs. 3.556 = 2.888).

Discussion

The present study has shown that increasing DHA from 0 (C) to 0.33% (L) increases DHA levels in all tissues studied, though the increases in retina, putamen, and amygdala did not reach statistical significance in the present study.

Dietary DHA at 0.3%, w/w normalized tissue DHA to levels found in breastfed neonates for all regions of the CNS except for the lobes of the cerebral cortex, where DHA increased compared to controls but was 87% to 90% of breastfed levels. A reasonable hypothesis is that higher DHA levels might further increase cortex DHA to breastfed levels. The present data show that precentral gyrus DHA increased by 24% from C to L, and 43% from C to L3. The additional increase from L to L3 of 19% was statistically significant, indicating that the greater DHA in the L3 formula was effective at increasing precentral gyrus DHA. Although the present study did not contain a breastfed control group, the magnitude of the increase was similar to the enhancement associated with the breastfed vs. term comparison. The inventors noted that the magnitude of the precentral gyrus DHA increase was less than two-fold, while the amount of DHA in the diet was tripled between L and L3. This observation indicates that the leveling off of tissue fatty acid concentrations in response to increases in dietary fatty acids, demonstrated in rats, was achieved in the primate brain at dietary DHA levels which were similar to the highest reported breastmilk levels.

The basal ganglia are a set of CNS organs that integrate and coordinate signals from the frontal cortex associated with executive function and motor coordination. The superior colliculus is a brainstem structure that controls saccades and also has cortical inputs, and the inferior colliculus is associated with the localization of sounds. Collectively, these CNS regions showed no significant difference in DHA between the L and L3 groups. In only the globus pallidus was the non-significant difference in L and L3 DHA of potential biological importance (11%); in the other tissues, DHA increased by less than 4% or decreased slightly. In part from this observation, it can be inferred that the necessarily modest statistical power of this primate study did not limit the ability to detect differences. These results are consistent with the conclusion that the cerebral cortex DHA is most sensitive to modest dietary DHA levels. Considering that DHA in the human CNS increases through two years of life, and that the cerebral cortex is quantitatively the largest CNS region, DHA demands may be important well beyond infancy.

Human and baboon breast milks contain the n-3 LCPUFA EPA and DPA at concentrations that are a substantive fraction of the DHA concentration. In adult humans, these LCPUFA are much more efficiently converted to DHA than α-linolenic acid (ALA). U.S. infant formulas contain negligible amounts of EPA and n-3 DPA because the source of n-3 LCPUFA, oil from the marine algae Crypthecodinium cohnii, does not contain these LCPUFAs. DHA levels that are higher than those in currently available formulas, and more similar to the L3 formula, may be indicated to make up for these minor n-3 LCPUFAs. Indeed, the study has found that n-3 DPA drops in most tissue in response to moderate DHA but rebounds at the L3 DHA level. The exception was retina in which n-3 DPA increased as DHA increased. EPA was at trace levels in the CNS.

In the liver, RBC, and plasma, ARA rose significantly in the L group and then achieves an intermediate value in the L3 group; an equivalent but non-significant pattern was found for the heart. The present results are consistent with previous data indicating that tissue ARA concentrations, particularly in the CNS are more refractory to formula ARA than DHA. No changes were found in the cerebral cortex, retina, putamen, caudate, and amygdala. However, L3 group ARA was reduced compared to control in the superior colliculus and compared to L in the globus pallidus.

Osbond acid (DPAn-6) is an elongation and 4-5 desaturation product of ARA that consistently rises in experimental n-3 fatty acid deficiency, and also drops in response to DHA supplementation in otherwise normal primates. DPAn-6 dropped in all tissues with increasing DHA, and in some tissues such as the cerebral cortex, L3 DPAn-3 values were a fraction of the C values. This decrease and the accompanying increase in DHA drove the DPA/DHA ratio decrease from the L to L3 groups.

These results indicate that DHA is more sensitive to dietary manipulations than ARA in most tissues. They show that cerebral cortex DHA increases with higher concentrations of DHA than are included in present commercial infant formulas, while not increasing the levels of DHA in basal ganglia and limbic system.

The data also provide support for the hypothesis that formula DHA at concentrations higher than presently used in formulas, but nevertheless well within the known range of human breast milk, normalizes CNS tissue composition closer to that of breastfeeding. Changes in tissue composition by themselves do not justify alteration of diet composition, and should be coupled to demonstrations of efficacy associated with improvements in functional outcomes.

These data also demonstrate that DHA and ARA (1) reciprocally regulate IL-15 expression in skeletal muscle and adipose tissue, which favor increased muscle mass and oppose excess adiposity; (2) reduce expression of the hepatic leptin receptor, thereby promoting greater satiating effects of circulating leptin; and (3) increase the expression of skeletal muscle adiponectin receptor, which enhances fatty acid oxidation and insulin sensitivity.

Supplementation with DHA and ARA also reduced hepatic de novo LCPUFA synthesis mediated via downregulation of sterol regulatory-binding protein-2 (SREBP2) with coordinated suppression of sterol-CoA desaturase (delta-9 desaturase), fatty acid desaturase (delta-5 desaturase), and fatty acid desaturase-2 (delta-6 desaturase). Suppression of sterol-CoA desaturase (SCD) suppresses the accumulation of omega-9 fatty acids in membranes to maintain proper phospholipids membrane composition. This is necessary for normal fetal and neonatal growth. Downregulation of SCD is consistent with the suppression of de novo fatty acid synthesis by DHA and ARA. The net result would be to reduce palmitoleate composition of triglycerides and adipocytes. In the present study, increasing DHA levels resulted in greater suppression of SCD mRNA levels, suggesting that higher levels of DHA more effectively suppress de novo lipogenesis and promote more favorable triglyceride and lipoprotein composition.

The net result of all of these actions leads to reduced de novo lipogenesis and enhanced fatty acid oxidation, improved insulin sensitivity, and improved leptin responsiveness, culminating in a metabolic environment unfavorable to the development of obesity.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. For example, while methods for the production of a commercially sterile liquid nutritional supplement made according to those methods have been exemplified, other uses are contemplated. Therefore, the spirit and scope of the appended claims should not be limited to the description of the preferred versions contained therein.

What is claimed is:

1. A method for preventing or treating obesity in a subject, the method comprising administering to the subject a therapeutically effective amount of DHA and ARA.

2. The method according to claim 1, wherein the subject is in need of such treatment or prevention.

3. The method according to claim 1, wherein the therapeutically effective amount of DHA is between about 15 mg per kg of body weight per day and 60 mg per kg of body weight per day.

4. The method according to claim 1, wherein the therapeutically effective amount of ARA is between about 20 mg per kg of body weight per day and 60 mg per kg of body weight per day.

5. The method according to claim 1, wherein the ratio of ARA:DHA by weight is from about 1:3 to about 9:1.

6. The method according to claim 1, wherein the ratio of ARA:DHA by weight is about 2:1.

7. The method according to claim 1, wherein the ratio of ARA:DHA by weight is about 1:1.5.

8. The method according to claim 1, wherein the subject is an infant.

9. The method according to claim 8, wherein the DHA and ARA are administered to the infant during the time period from birth until the infant is about one year of age.

10. The method according to claim 8, wherein the DHA and ARA are administered to the infant in an infant formula.

11. The method according to claim 10, wherein the infant formula comprises DHA in an amount of from about 15 mg to about 60 mg per 100 kcal infant formula.

12. The method according to claim 10, wherein the infant formula comprises ARA in an amount of from about 25 mg to about 40 mg per 100 kcal infant formula.

13. A method for increasing the lean muscle mass and decreasing the adipose tissue of a subject, the method comprising administering to the subject a therapeutically effective amount of DHA and ARA.

14. A method for increasing the lean muscle mass and decreasing the adipose tissue of an infant, the method comprising administering to the infant a therapeutically effective amount of DHA and ARA, wherein the ratio of ARA:DHA by weight is from about 1:3 to about 9:1.

15. The method according to claim 14, wherein the ratio of ARA:DHA by weight is about 2:1.

16. The method according to claim 14, wherein the ratio of ARA:DHA by weight is about 1:1.5.

17. A method for upregulating the expression of IL-15 in a subject's skeletal muscle, the method comprising administering to the subject a therapeutically effective amount of DHA and ARA.

18. A method for downregulating the expression of IL-15 in a subject's subcutaneous adipose tissue, the method comprising administering to the subject a therapeutically effective amount of DHA and ARA.

19. A method for upregulating the expression of adiponectin in a subject's skeletal muscle, the method comprising administering to the subject a therapeutically effective amount of DHA and ARA.

20. A method for downregulating the expression of the hepatic leptin receptor in a subject, the method comprising administering to the subject a therapeutically effective amount of DHA and ARA.

21. A method for preventing or treating obesity in an infant, the method comprising administering to the infant DHA.

22. A method for preventing or treating obesity in an infant, the method comprising administering to the infant ARA.

23. A method for preventing or treating obesity in a child, the method comprising administering to the child DHA.

24. The method according to claim 23, wherein the child is between the ages of one and six years of age.

25. The method according to claim 23, wherein the child is between the ages of about seven and twelve years of age.

26. The method according to claim 23 additionally comprising administering ARA to the child.

27. A method for preventing or treating obesity in a child, the method comprising administering to the child ARA.
